(19) United States
(12) Patent Application Publication
(10) Pub. No.: US 2008/0085888 A1
(43) Pub. Date: Apr. 10, 2008

(54) Therapeutic Combinations

(76) Inventors: Scott R. Breining, Winston-Salem, NC (US); John L. Evenden, Wilmington, DE (US); Edwin Johnson, Wilmington, DE (US); Kristen G. Jordan, Clemmons, NC (US); Sharon R. Letchworth, Kernersville, NC (US); Craig H. Miller, Winston-Salem, NC (US); Ladislav Mrzljak, Wilmington, DE (US); James K. Wamsley, Pfafftown, NC (US); Dan Widzowski, Wilmington, DE (US); Yun-De Xiao, Clemmons, NC (US)

Correspondence Address: Pepper Hamilton LLP, 400 Berwyn Park, 899 Cassatt Road, Berwyn, PA, 19312-1183, US

(21) Appl. No.: 11/855,175

(22) Filed: Sep. 14, 2007

Related U.S. Application Data
(60) Provisional application No. 60/844,759, filed on Sep. 15, 2006.

(51) Int. Cl. A61K 31/44; A61K 31/451; A61K 31/554; A61P 25/18

(52) U.S. Cl. 514/211.13; 514/327; 514/357

(57) ABSTRACT

The present invention provides a combination of (a) an antipsychotic and (b) an alpha4/beta2 (α4β2)-neuronal nicotinic receptor agonist. The invention further relates to pharmaceutical compositions comprising said combination and to the use of the combination in therapy. The invention further relates to a kit comprising the combination and use of said kit in therapy.

### CROSS-REFERENCE TO RELATED APPLICATIONS

This application claims priority to U.S. provisional application Ser. No. 60/844,759 filed Sep. 15, 2006, which is incorporated herein by reference in its entirety.

### FIELD OF THE INVENTION

The present invention is directed, in part, to combinations of (a) an antipsychotic and (b) an alpha-4/beta-2 (α4β2)-neuronal nicotinic receptor agonist. The invention further relates to pharmaceutical compositions comprising said combination and to the use of the combination in therapy. The invention further relates to a kit comprising the combination and use of said kit in therapy.

### BACKGROUND OF THE INVENTION

Exemplary conventional antipsychotics may include, but are not limited to, chlorpromazine, haloperidol, flupenthixol and perphenazine. Examples of atypical antipsychotics include, but are not limited to, clozapine, risperidone, olanzapine, quetiapine, aripiprazole, ziprasidone, amisulpride, sulpride, zotepine, sertindole, paliperidone, bifeprunox, and asenapine.

Atypical antipsychotics offer several clinical benefits over the conventional antipsychotics. The distinct advantages over traditional antipsychotic medications include greater improvement in negative symptoms, such as social withdrawal, and lower risk of Parkinsonian side effects and tardive dyskinesia.

Quetiapine, the international nonproprietary name for 11-[4-[2-(2-hydroxyethoxy)ethyl]-1-piperazinyl]dibenzo[b,f][1,4]thiazepine, is an atypical antipsychotic and is currently on the market as Seroquel for 1) treatment of schizophrenia, 2) adjunctive therapy with mood stabilizers (lithium or divalproex) in the treatment of acute manic episodes associated with bipolar I disorders, 3) monotherapy in the treatment of acute manic episodes associated with bipolar I disorder, and 4) treatment of major depressive episodes associated with bipolar disorder.

Quetiapine and its pharmaceutically acceptable salts are described in U.S. Pat. No. 4,879,288, which is incorporated herein by reference in its entirety. A preparation of these compounds is also described in said U.S. patent.

Cognitive dysfunction is also an integral feature of depression and schizophrenia (Psychol Med. 24:829 (1994); Am. J. Psychiatry 161:25 (2004)). Significant deficits have been found in a range of neuropsychological measures covering aspects of language function, memory, both recall and recognition, attention and behavioral regulation.

The neuronal nicotinic receptor agonists of the present invention are those compounds having agonist or partial agonist activity against the alpha-4/beta-2, or α4/β2, receptor (α4β2-neuronal nicotinic receptor agonist). Particular neuronal nicotinic receptor agonists useful in the combination of the present invention are those described in U.S. Pat. Nos. 6,603,011 and 6,958,399, each of which is hereby incorporated by reference in its entirety. Particular neuronal nicotinic receptor agonists are compounds N-methyl-5-[3-(5-isopropoxypyridin)yl]-4-penten-2-amine, (4E)-N-methyl-5-[3-(5-isopropoxypyridin)yl]-4-penten-2-amine and (2S)-(4E)-N-methyl-5-[3-(5-isopropoxypyridin)yl]-4-penten-2-amine, metabolites or prodrugs and pharmaceutically acceptable salts, solvates or solvated salts of any of the foregoing. The preparation of these compounds is described in said U.S. patents. These compounds modulate nicotinic receptors in the patient's brain. As such, the compounds have the ability to express nicotinic pharmacology, and in particular, to act as neuronal nicotinic receptor agonists.

The α4β2-neuronal nicotinic receptor agonists can be used to treat those types of conditions and disorders for which other types of nicotinic compounds have been proposed as therapeutics. The neuronal nicotinic receptor agonists are useful in the treatment of a variety of CNS disorders including, but not limited to, neurodegenerative disorders, neuropsychiatric disorders, neurologic disorders, addictions, disorders attributed to or associated with neurotransmitter system dysfunction, CNS disorders attributed to a cholinergic deficiency, a dopaminergic deficiency, an adrenergic deficiency and/or a serotonergic deficiency.

### SUMMARY OF THE INVENTION

In one embodiment, the present invention relates to the combination of (a) a first therapeutic agent, which is an antipsychotic agent and (b) a second therapeutic agent, which is (2S)-(4E)-N-methyl-5-[3-(5-isopropoxypyridin)yl]-4-penten-2-amine. Particularly, the synergistic combination of (a) and (b).

The combinations described herein are contemplated to provide synergistic or additive effects in treating psychiatric disorders; particularly, cognitive impairment disorders in psychotic disorders. Described combinations are contemplated to provide symptomatic relief of psychiatric disorders; particularly, cognitive impairment disorders in psychotic disorders.

Compositions and methods described herein are contemplated to offer advantages over previous methods for treating neuropsychiatric disorders. The method of treatment described herein is contemplated to enhance the effect of an antipsychotic when taken in combination with a neuronal nicotinic receptor agonist and therefore, in one aspect, permit reduced quantities of these agents to be used and, therefore, permit improved management of cognitive impairment, disease symptoms and disease-related side effects.

The combinations are contemplated to have a reduction of drug-induced extrapyramidal symptoms (EPS).

Further the combinations are contemplated to have fewer side effects, such as a reduction in the quetiapine-induced sedation.

Some have suggested that neuronal nicotinic agonists either worsen or have no effect on haloperidol-induced catalepsy (Sanberg, P., et al., Pharm. Biochem. Behav. 46: 303-307, (1993), and Levin, E., et al., Drug Devel. Res., 47:90-96 (1999)). However, the present invention comptemplates that the combinations disclosed herein reduce the haloperidol-induced catalepsy.

The described combinations are also contemplated to complement sedatives and mood stabilizers such as lithium, as well as prophylactically address progression of psychotic conditions and/or decline of cognitive function in psychotic conditions.

A further advantage of this synergistic effect may be a quicker onset of the therapeutic effect than that of the single compounds.

Other features and advantages will be apparent from the following detailed description and from the claims.

In particular, the present invention provides combinations comprising (a) a first therapeutic agent, which is an antipsychotic agent and (b) a second therapeutic agent, which is (2S)-(4E)-N-methyl-5-[3-(5-isopropoxypyridin)yl]-4-penten-2-amine or pharmaceutically acceptable salts thereof. In some embodiments, the first therapeutic agent is quetiapine or pharmaceutically acceptable salts thereof.

The present invention also provides pharmaceutical compositions comprising a combination comprising (a) a first therapeutic agent, which is an antipsychotic agent and (b) a second therapeutic agent, which is (2S)-(4E)-N-methyl-5-[3-(5-isopropoxypyridin)yl]-4-penten-2-amine, or pharmaceutically acceptable salts thereof, together with a pharmaceutically acceptable vehicle, carrier or diluent. In some embodiments, the first therapeutic agent is quetiapine or pharmaceutically acceptable salts thereof.

The present invention also provides kits comprising (a) a dosage unit of a first therapeutic agent, which is, an antipsychotic agent and (b) a dosage unit of a second therapeutic agent, which is (2S)-(4E)-N-methyl-5-[3-(5-isopropoxypyridin)yl]-4-penten-2-amine, or pharmaceutically acceptable salts thereof, optionally with instructions for use. In some embodiments, the first therapeutic agent is quetiapine or pharmaceutically acceptable salts thereof.

The present invention also provides methods for treating cognitive impairment and/or psychotic disorder in a subject in need thereof comprising administering simultaneously, sequentially or separately, to said subject a therapeutically effective amount of (a) a first therapeutic agent, which is an antipsychotic agent and (b) a second therapeutic agent, which is (2S)-(4E)-N-methyl-5-[3-(5-isopropoxypyridin)yl]-4-penten-2-amine, or pharmaceutically acceptable salts thereof. In some embodiments, the psychotic disorder or condition is selected from the group consisting of schizophrenia, schizophreniform disorder, schizoaffective disorder, delusional disorder, brief psychotic disorder, treatment-resistant shared psychotic disorder, psychotic disorder due to a medical condition. In some embodiments, the first therapeutic agent, which is an antipsychotic agent and the second therapeutic agent, which is (2S)-(4E)-N-methyl-5-[3-(5-isopropoxypyridin)yl]-4-penten-2-amine, or pharmaceutically acceptable salts thereof are administered simultaneously, sequentially or separately, to the subject in a pharmaceutical composition additionally comprising a pharmaceutically acceptable vehicle, carrier or diluent.

The present invention also provides methods for improving the antipsychotic effect of quetiapine in a subject in need thereof comprising administering simultaneously, sequentially or separately, to said subject ((a) a first therapeutic agent, which is an antipsychotic agent and (b) a second therapeutic agent, which is (2S)-(4E)-N-methyl-5-[3-(5-isopropoxypyridin)yl]-4-penten-2-amine, or pharmaceutically acceptable salts thereof.

The present invention also provides methods of increasing dopamine release in prefrontal cortex in a subject in need thereof comprising administering simultaneously, sequentially or separately, to said subject (a) a first therapeutic agent, which is an antipsychotic agent and (b) a second therapeutic agent, which is (2S)-(4E)-N-methyl-5-[3-(5-isopropoxypyridin)yl]-4-penten-2-amine, or pharmaceutically acceptable salts thereof.

The present invention also provides methods of reducing sedative side effects of quetiapine in a subject in need thereof comprising administering simultaneously, sequentially or separately, to said subject a therapeutically effective amount of (a) a first therapeutic agent, which is an antipsychotic agent and (b) a second therapeutic agent, which is (2S)-(4E)-N-methyl-5-[3-(5-isopropoxypyridin)yl]-4-penten-2-amine, or pharmaceutically acceptable salts thereof.

In some embodiments, the first therapeutic agent is quetiapine or pharmaceutically acceptable salts. In some embodiments, the kit described herein is used. In some embodiments, the dose of quetiapine is between 5 and 50 mg/kg. In some embodiments, the dose of quetiapine is between 10 and 40 mg/kg. In some embodiments, the dose of (2S)-(4E)—N-methyl-5-[3-(5-isopropoxypyridin)yl]-4-penten-2-amine is between 0.001 and 0.5 mg/kg. In some embodiments, the dose of (2S)-(4E)-N-methyl-5-[3-(5-isopropoxypyridin)yl]-4-penten-2-amine is between 0.01 and 0.3 mg/kg.

The present invention also provides methods of inhibiting acoustic startle in a subject in need thereof comprising administering simultaneously, sequentially or separately, to said subject (a) a first therapeutic agent, which is an antipsychotic agent and (b) a second therapeutic agent, which is (2S)-(4E)-N-methyl-5-[3-(5-isopropoxypyridin)yl]-4-penten-2-amine, or pharmaceutically acceptable salts thereof.

The present invention also provides methods of enhancing quetiapine-mediated reduction of phencyclidine-induced disruption of prepulse inhibition in a subject in need thereof comprising administering simultaneously, sequentially or separately, to said subject (a) a first therapeutic agent, which is an antipsychotic agent and (b) a second therapeutic agent, which is (2S)-(4E)-N-methyl-5-[3-(5-isopropoxypyridin)yl]-4-penten-2-amine, or pharmaceutically acceptable salts thereof. In some of embodiments, the first therapeutic agent is quetiapine or pharmaceutically acceptable salts. In some embodiments, the kit described herein is used. In some embodiments, the dose of quetiapine is between 1 and 25 mg/kg. In some embodiments, the dose of quetiapine is between 5 and 20 mg/kg. In some embodiments, the dose of (2S)-(4E)-N-methyl-5-[3-(5-isopropoxypyridin)yl]-4-penten-2-amine is between 0.001 and 0.05 mg/kg. In some embodiments, the dose of (2S)-(4E)-N-methyl-5-[3-(5-isopropoxypyridin)yl]-4-penten-2-amine is between 0.01 and 0.03 mg/kg.

The present invention also provides methods of reducing haloperidol-induced catalepsy in a subject in need thereof comprising administering simultaneously, sequentially or separately, to said subject (a) a first therapeutic agent, which is haloperidol (b) a second therapeutic agent, which is (2S)-(4E)-N-methyl-5-[3-(5-isopropoxypyridin)yl]-4-penten-2-amine, or pharmaceutically acceptable salts thereof.

The present invention also provides combinations comprising (a) a first therapeutic agent, which is an antipsychotic agent and (b) a second therapeutic agent, which is an α4β2-selective neuronal nicotinic receptor agonist, or pharmaceutically acceptable salts thereof.

The present invention also provides pharmaceutical compositions comprising a combination comprising (a) a first therapeutic agent, which is an antipsychotic agent and (b) a second therapeutic agent, which is an α4β2-selective neuronal nicotinic receptor agonist, or pharmaceutically acceptable salts thereof, together with a pharmaceutically acceptable vehicle, carrier or diluent.

The present invention also provides methods for treating cognitive impairment and psychotic disorder in a subject in need thereof comprising administering simultaneously, sequentially or separately, to said subject (a) an amount of a first therapeutic agent, which is an antipsychotic agent or pharmaceutically acceptable salts thereof and (b) an amount of a second therapeutic agent, which is an α4β2 selective neuronal nicotinic receptor agonist, or pharmaceutically acceptable salts thereof.

### BRIEF DESCRIPTION OF THE DRAWINGS

FIG. 1 shows effects of Compound b alone on spontaneous locomotor activity (sLMA) in rats at doses from 0.006 to 0.19 mg/kg p.o. (bars represent group mean ±SD; n=8 per treatment; *=p<0.05, and NS=not significant with respect to vehicle (v)).

FIG. 2 shows effects of Compound b at 0.19 mg/kg or quetiapine at 40 mg/kg alone or in combination on sLMA in rats (bars represent group mean ±SD; n=8 per treatment; *=p<0.05 with respect to vehicle+vehicle (v+v)).

FIG. 3 shows effects of quetiapine alone (10, 20, 40 or 60 mg/kg s.c.) on D-amphetamine-induced locomotor activity (bars represent group mean ±SD; n=8 per treatment; *=p<0.05 and **=p<0.01 compared to vehicle+D-amphetamine (V+A)).

FIG. 4 shows effects of quetiapine 10 mg/kg s.c. or Compound b alone or in combination on D-amphetamine-induced locomotor activity (bars represent group mean ±SD; n=12 per treatment; *=p<0.05 compared to vehicle+D-amphetamine (V+V+A)).

FIG. 5 shows effects of compound b on haloperidol-induced catalepsy (n=10 per treatment; *=p<0.05 compared to vehicle+haloperidol).

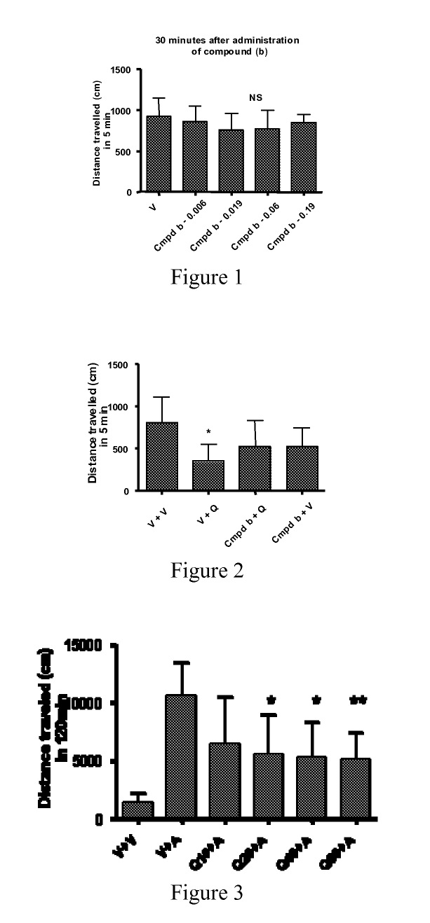

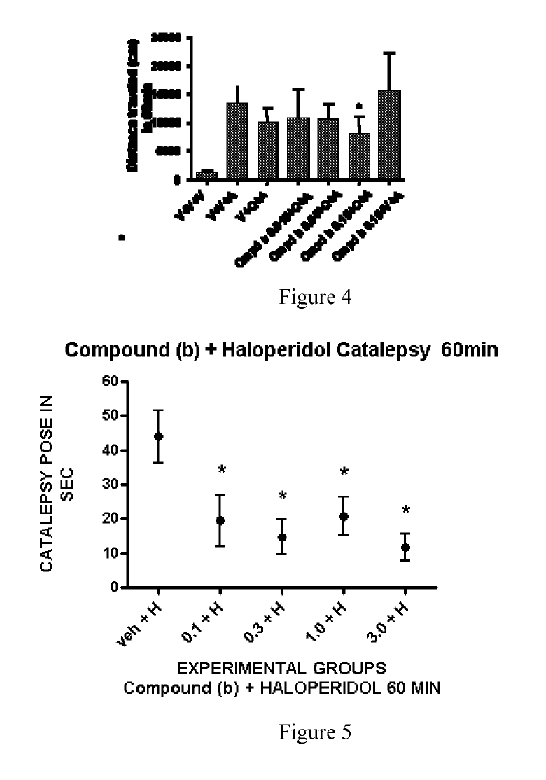

### DESCRIPTION OF EMBODIMENTS

### Combinations

A first aspect of the invention relates to a combination comprising (a) a first therapeutic agent, which is an antipsychotic agent and (b) a second therapeutic agent, which is an α4β2-neuronal nicotinic receptor agonist. In one embodiment of the invention, the combination comprises the group of compounds (a) and (b) as defined below.

One embodiment relates to a combination wherein the antipsychotic agent (a) is an atypical antipsychotic agent.

In a further embodiment of the invention, the first therapeutic agent (a) comprises of compounds selected from chlorpromazine, haloperidol, flupenthixol and perphenazine. Examples of atypical antipsychotics include, but are not limited to, clozapine, risperidone, olanzapine, quetiapine, aripiprazole, ziprasidone, amisulpride, sulpride, zotepine, sertindole, paliperidone, bifeprunox, and asenapine, metabolites or prodrugs and pharmaceutically acceptable salts, solvates or solvated salts thereof.

In another embodiment of the invention, the first therapeutic agent (a) is quetiapine and pharmaceutically acceptable salts, solvates or solvated salts thereof.

In one embodiment of the invention, the second therapeutic agent (b) comprises compounds which are neuronal nicotinic receptor agonists having binding action at the α4β2-nicotinic acetylcholine receptor, such as compounds described in U.S. Pat. Nos. 6,603,011 and 6,958,399. In one embodiment, the second therapeutic agent (b) is (2S)-(4E)-N-methyl-5-[3-(5-isopropoxypyridin)yl]-4-penten-2-amine, metabolites or prodrugs and pharmaceutically acceptable salts, solvates or solvated salts thereof.

It will be recognized by those skilled in the art in light of this disclosure that any neuronal nicotinic receptor agonists having binding action at the α4β2-nicotinic acetylcholine receptor may be useful in the combinations, pharmaceutical compositions, methods and kits described herein.

A further embodiment of the invention relates to a combination comprising (a) a first therapeutic agent, which is an antipsychotic agent and (b) a second therapeutic agent, which is (2S)-(4E)-N-methyl-5-[3-(5-isopropoxypyridin)yl]-4-penten-2-amine or pharmaceutically acceptable salts thereof.

Another embodiment of the invention relates to a combination comprising (a) a first therapeutic agent, which is quetiapine or pharmaceutically acceptable salts thereof and (b) a second therapeutic agent, which is (2S)-(4E)-N-methyl-5-[3-(5-isopropoxypyridin)yl]-4-penten-2-amine or pharmaceutically acceptable salts thereof.

Pharmaceutical Compositions

Another aspect of the invention relates to a pharmaceutical composition comprising a combination comprising (a) an antipsychotic and (b) an α4β2-neuronal nicotinic receptor agonist, together with a pharmaceutically acceptable vehicle, carrier or diluent.

One embodiment of the invention relates to one pharmaceutical composition comprising both agents (a) and (b). Another embodiment relates to a two separate pharmaceutical compositions, one for agent (a) and one for agent (b).

One embodiment relates to a pharmaceutical composition as described above wherein the first therapeutic agent (a) comprises the group of compounds selected from chlorpromazine, haloperidol, flupenthixol, and perphenazine. Examples of atypical antipsychotics include but are not limited to clozapine, risperidone, olanzapine, quetiapine, aripiprazole, ziprasidone, amisulpride, sulpride, zotepine, sertindole, paliperidone, bifeprunox, and asenapine, metabolites or prodrugs and pharmaceutically acceptable salts, solvates or solvated salts thereof.

A further embodiment relates to a pharmaceutical composition wherein the antipsychotic agent is quetiapine, metabolites or prodrugs and pharmaceutically acceptable salts, solvates or solvated salts thereof.

Another embodiment relates to a pharmaceutical composition as described above wherein the second therapeutic agent is (2S)-(4E)-N-methyl-5-[3-(5-isopropoxypyridin)yl]-4-penten-2-amine, metabolites or prodrugs and pharmaceutically acceptable salts, solvates or solvated salts thereof.

One embodiment relates to a pharmaceutical composition comprising a combination comprising (a) a first therapeutic agent, which is an antipsychotic agent and (b) a second therapeutic agent, which is (2S)-(4E)-N-methyl-5-[3-(5-isopropoxypyridin)yl]-4-penten-2-amine, or pharmaceutically acceptable salts thereof, together with a pharmaceutically acceptable vehicle, carrier or diluent.

Another embodiment relates to a pharmaceutical composition comprising a combination comprising (a) a first therapeutic agent, which is quetiapine or pharmaceutically acceptable salts thereof and (b) a second therapeutic agent, which is (2S)-(4E)-N-methyl-5-[3-(5-isopropoxypyridin)yl]-4-penten-2-amine, or pharmaceutically acceptable salts thereof, together with a pharmaceutically acceptable vehicle, carrier or diluent.

For use in medicine, pharmaceutically acceptable salts may be useful in the preparation of the compounds used in the combination of the invention. Suitable pharmaceutically acceptable salts of the compounds described herein include acid addition salts which may, for example, be formed by mixing a solution of the compound according to the invention with a solution of a pharmaceutically acceptable acid such as hydrochloric acid, sulfuric acid, methanesulphonic acid, and fumaric acid. Furthermore, where the compounds carry an acidic moiety, suitable pharmaceutically acceptable salts thereof may include alkali metal salts, e.g. sodium or potassium salts; alkaline earth metal salts, e.g. calcium or magnesium salts; and salts formed with suitable organic ligands, e.g. quaternary ammonium salts.

The expression “pharmaceutically acceptable salts” includes both pharmaceutically acceptable acid addition salts and pharmaceutically acceptable cationic salts and also includes solvates, hydrates or solvated or hydrated salts thereof. The expression “pharmaceutically acceptable cationic salts” is intended to define but is not limited to such salts as the alkali metal salts, (e.g. sodium and potassium), alkaline earth metal salts (e.g., calcium and magnesium), aluminum salts, ammonium salts, and salts with organic amines such as benzathine (N,N′-dibenzylethylenediamine) and choline. The expression “pharmaceutically acceptable acid addition salts” is intended to define but is not limited to such salts as the hydrochloride, hydrobromide and sulfate.

The pharmaceutically acceptable cationic salts containing free carboxylic acids can be readily prepared by reacting the free acid form of with an appropriate base. Typical bases are sodium hydroxide, sodium methoxide, and sodium ethoxide.

The pharmaceutically acceptable acid addition salts containing free amine groups can be readily prepared by reacting the free base form with the appropriate acid.

The salts of the antipsychotic compound may be prepared according to the process described in U.S. Pat. No. 4,879,288 and are preferably pharmaceutically acceptable salts, but other salts may also be prepared. A preferred salt is the hemi-fumarate salt. Suitable salts for the α4β2-neuronal nicotinic receptor agonist, may be, but are not limited to, tartaric acid, hydroxybenzoic acid, phosphoric acid, edisylic acid (1,2-ethanedisulfonic acid), citric acid, orotic acid (uracil-6-carboxylic acid), R-mandelic acid, sulfuric acid, 1,5-naphthalenedisulfonic acid, D-aspartic acid and lysine monohydrochloride salts.

An α4β2-neuronal nicotinic receptor agonist such as (2S)-(4E)-N-methyl-5-[3-(5-isopropoxypyridin)yl]-4-penten-2-amine, may be co-administered simultaneously or may be administered separately or sequentially in any order, or as a single pharmaceutical composition with an atypical antipsychotic such as quetiapine to produce a synergistic benefit over and above that obtained by administration of either compound alone.

The composition may be administered in a standard manner such as orally, parenterally, transmucosally (e.g., sublingually or via buccal administration), topically, transdermally, rectally, via inhalation (e.g., nasal or deep lung inhalation). Parenteral administration includes, but is not limited to, intravenous, intraarterial, intraperitoneal, subcutaneous or intramuscular.

For buccal administration, the composition can be in the form of tablets or lozenges formulated in conventional manner. For example, tablets and capsules for oral administration can contain conventional excipients such as binding agents (e.g., syrup, acacia, gelatin, sorbitol, tragacanth, mucilage of starch or polyvinylpyrrolidone), fillers (e.g., lactose, sugar, microcrystalline cellulose, maize-starch, calcium phosphate or sorbitol), lubricants (e.g., magnesium stearate, stearic acid, talc, polyethylene glycol or silica), disintegrants (e.g., potato starch or sodium starch glycollate), or wetting agents (e.g., sodium lauryl sulfate). Tablets may be coated according to methods well known in the art.

Such compositions can also be formulated as suppositories for rectal administration, e.g., containing conventional suppository bases, such as cocoa butter or other glycerides. Compositions for inhalation typically can be provided in the form of a solution, suspension, or emulsion that can be administered as a dry powder or in the form of an aerosol using a conventional propellant, such as dichlorodifluoromethane or trichlorofluoromethane. Typical topical and transdermal formulations comprise conventional aqueous or nonaqueous vehicles, such as eye drops, creams, ointments, lotions, and pastes, or are in the form of a medicated plaster, patch, or membrane.

Additionally, compositions described herein can be formulated for parenteral administration by injection or continuous infusion. Formulations for injection can be in the form of suspensions, solutions, or emulsions in oily or aqueous vehicles, and can contain formulation agents, such as suspending, stabilizing, and/or dispersing agents. Alternatively, the active ingredient can be in powder form for constitution with a suitable vehicle (e.g., sterile, pyrogen-free water) before use.

A composition in accordance with the present invention also can be formulated as a depot formulation. Such long acting formulations can be administered by implantation (e.g., subcutaneously or intramuscularly) or by intramuscular injection. Accordingly, the compounds of the invention can be formulated with suitable polymeric or hydrophobic materials (e.g., an emulsion in an acceptable oil), ion exchange resins, or as sparingly soluble derivatives (e.g., a sparingly soluble salt).

For oral administration a pharmaceutical composition can take the form of solutions, suspensions, tablets, pills, capsules, powders, and the like. Tablets containing various excipients such as sodium citrate, calcium carbonate and calcium phosphate are employed along with various disintegrants such as starch, and preferably potato or tapioca starch, and certain complex silicates, together with binding agents such as polyvinylpyrrolidone, sucrose, gelatin and acacia. Additionally, lubricating agents such as magnesium stearate, sodium lauryl sulfate, and talc may be used to form tablets. Solid compositions of a similar type are also employed as fillers in soft and hard-filled gelatin capsules; preferred materials in this connection also include lactose or milk sugar as well as high molecular weight polyethylene glycols.

Alternatively, the composition described herein can be incorporated into oral liquid formulations such as aqueous or oily suspensions, solutions, emulsions, syrups, or elixirs, for example. Moreover, formulations containing these compounds can be presented as a dry product for constitution with water or other suitable vehicle before use. Such liquid formulations can contain conventional additives, such as suspending agents, such as sorbitol syrup, synthetic and natural gums such as tragacanth, acacia, alginate, dextran, sodium carboxymethylcellulose, methylcellulose, polyvinyl-pyrrolidone or gelatin, glucose/sugar syrup, gelatin, hydroxyethylcellulose, hydroxypropylmethylcellulose, aluminum stearate gel, emulsifying agents, such as lecithin, sorbitan monooleate, or acacia; nonaqueous vehicles (which can include edible oils), such as almond oil, fractionated coconut oil, oily esters, propylene glycol, and ethyl alcohol; and preservatives, such as methyl or propyl p-hydroxybenzoate and sorbic acid. The liquid forms in which the compositions described herein may be incorporated for administration orally or by injection include aqueous solutions, suitably flavored syrups, aqueous or oil suspensions, and flavored emulsions with edible oils such as cottonseed oil, sesame oil, coconut oil or peanut oil, as well as elixirs and similar pharmaceutical vehicles.

When aqueous suspensions and/or elixirs are desired for oral administration, the compounds described herein can be combined with various sweetening agents, flavoring agents, coloring agents, emulsifying agents and/or suspending agents, as well as such diluents as water, ethanol, propylene glycol, glycerin and various like combinations thereof. Suitable dispersing or suspending agents for aqueous suspensions include synthetic and natural gums such as tragacanth, acacia, alginate, dextran, sodium carboxymethylcellulose, methylcellulose, polyvinyl-pyrrolidone or gelatin.

The combinations described herein can also be administered in a controlled release formulation (definition) such as a slow release or a fast release formulation. Such controlled release formulations of the combinations described herein may be prepared using methods well known to those skilled in the art. The method of administration will be determined, by the attendant physician or other person skilled in the art after an evaluation of the patient's condition and requirements.

Kits

Another aspect of the invention relates to a kit comprising a dosage unit of a first therapeutic agent, which is an atypical antipsychotic and a dosage unit of a second therapeutic agent, which is an α4β2-neuronal nicotinic receptor agonist, optionally with instructions for use.

One embodiment relates to a kit as described above wherein the first therapeutic agent (a) comprises the group of compounds selected from chlorpromazine, haloperidol, flupenthixol, and perphenazine. Examples of atypical antipsychotics include, but are not limited to, clozapine, risperidone, olanzapine, quetiapine, aripiprazole, ziprasidone, amisulpride, sulpride, zotepine, sertindole, paliperidone, bifeprunox and asenapine, metabolites or prodrugs and pharmaceutically acceptable salts, solvates or solvated salts thereof.

A further embodiment relates to a kit wherein the antipsychotic agent is quetiapine, metabolites or prodrugs and pharmaceutically acceptable salts, solvates or solvated salts thereof.

Another embodiment relates to a kit as described above wherein the second therapeutic agent is (2S)-(4E)-N-methyl-5-[3-(5-isopropoxypyridin)yl]-4-penten-2-amine, metabolites or prodrugs and pharmaceutically acceptable salts, solvates or solvated salts thereof.

One embodiment relates to a kit comprising (a) a first therapeutic agent, which is an antipsychotic agent and (b) a second therapeutic agent, which is (2S)-(4E)-N-methyl-5-[3-(5-isopropoxypyridin)yl]-4-penten-2-amine, or pharmaceutically acceptable salts thereof.

Yet a further embodiment relates to a kit comprising (a) a dosage unit of a first therapeutic agent, which is quetiapine or pharmaceutically acceptable salts thereof and (b) a dosage unit of a second therapeutic agent, which is (2S)-(4E)-N-methyl-5-[3-(5-isopropoxypyridin)yl]-4-penten-2-amine, or pharmaceutically acceptable salts thereof, optionally with instructions for use.

Methods of Treatment

Another aspect of the invention relates to the use of the combination of the present invention in therapy.

One embodiment relates to a method for treating psychiatric disorders; particularly, cognitive impairment disorders in psychotic disorders in a subject in need thereof comprising administering simultaneously, sequentially or separately, to said subject (a) a first therapeutic agent, which is an antipsychotic agent and (b) a second therapeutic agent, which is (2S)-(4E)-N-methyl-5-[3-(5-isopropoxypyridin)yl]-4-penten-2-amine, or pharmaceutically acceptable salts thereof.

In another embodiment, the psychotic disorder or condition is selected from the group consisting of schizophrenia, schizophreniform disorder, schizoaffective disorder, delusional disorder, brief psychotic disorder, treatment-resistant shared psychotic disorder, psychotic disorder due to a medical condition, and psychotic disorder not otherwise specified.

Another embodiment relates to a method for reducing sedative side effects of quetiapine in a subject in need thereof comprising administering simultaneously, sequentially or separately to said subject (a) a first therapeutic agent, which is an antipsychotic agent and (b) a second therapeutic agent, which is (2S)-(4E)-N-methyl-5-[3-(5-isopropoxypyridin)yl]-4-penten-2-amine, or pharmaceutically acceptable salts thereof.

A further embodiment relates to a method for improving the antipsychotic effect of quetiapine in a subject in need thereof comprising administering simultaneously, sequentially or separately, to said subject (a) a first therapeutic agent, which is an antipsychotic agent and (b) a second therapeutic agent, which is (2S)-(4E)-N-methyl-5-[3-(5-isopropoxypyridin)yl]-4-penten-2-amine, or pharmaceutically acceptable salts thereof.

Another embodiment relates to a method of reducing extrapyramidal symptoms (EPS) in a subject in need thereof comprising administering simultaneously, sequentially or separately, to said subject (a) a first therapeutic agent, which is an antipsychotic agent and (b) a second therapeutic agent, which is (2S)-(4E)-N-methyl-5-[3-(5-isopropoxypyridin)yl]-4-penten-2-amine, or pharmaceutically acceptable salts thereof.

One embodiment relates to a method of inhibiting acoustic startle in a subject in need thereof comprising administering simultaneously, sequentially or separately, to said subject (a) a first therapeutic agent, which is an antipsychotic agent and (b) a second therapeutic agent, which is (2S)-(4E)-N-methyl-5-[3-(5-isopropoxypyridin)yl]-4-penten-2-amine, or pharmaceutically acceptable salts thereof.

Another embodiment relates to a method of enhancing quetiapine-mediated reduction of phencyclidine-induced disruption of prepulse inhibition in a subject in need thereof comprising administering simultaneously, sequentially or separately, to said subject (a) a first therapeutic agent, which is an antipsychotic agent and (b) a second therapeutic agent, which is (2S)-(4E)-N-methyl-5-[3-(5-isopropoxypyridin)yl]-4-penten-2-amine, or pharmaceutically acceptable salts thereof.

A further embodiment relates to a method of increasing dopamine release in prefrontal cortex in a subject in need thereof comprising administering simultaneously, sequentially or separately, to said subject (a) a first therapeutic agent, which is an antipsychotic agent and (b) a second therapeutic agent, which is (2S)-(4E)-N-methyl-5-[3-(5-isopropoxypyridin)yl]-4-penten-2-amine, or pharmaceutically acceptable salts thereof.

Yet another embodiment relates to a method of reducing haloperidol-induced catalepsy in a subject in need thereof comprising administering simultaneously, sequentially or separately, to said subject (a) a first therapeutic agent, which is haloperidol (b) a second therapeutic agent, which is (2S)-(4E)-N-methyl-5-[3-(5-isopropoxypyridin)yl]-4-penten-2-amine, or pharmaceutically acceptable salts thereof.

A further embodiment relates to said methods wherein, agent (a) and agent (b), are administered simultaneously, sequentially or separately, to the subject in a pharmaceutical composition additionally comprising a pharmaceutically acceptable vehicle, carrier or diluent, by a method selected from the group consisting of orally, parenterally, transmucosally (e.g., sublingually or via buccal administration), topically, transdermally, rectally, via inhalation (e.g., nasal or deep lung inhalation). Parenteral administration includes, but is not limited to, intravenous, intraarterial, intraperitoneal, subcutaneous, intradermal, intramuscular, intrathecal or via a high pressure technique.

One embodiment of the invention relates to a method of treating psychiatric disorders; particularly, cognitive impairment disorders in psychotic disorders in a subject in need thereof using the kit as described above.

One embodiment of the invention relates to the use of the combination comprising (a) a first therapeutic agent, which is an antipsychotic agent and (b) a second therapeutic agent, which is (2S)-(4E)-N-methyl-5-[3-(5-isopropoxypyridin)yl]-4-penten-2-amine, or pharmaceutically acceptable salts thereof, for the manufacturing of a medicament for use simultaneously, sequentially or separately, in therapy.

Another embodiment of the invention relates to the use of the combination comprising (a) a first therapeutic agent, which is an antipsychotic agent and (b) a second therapeutic agent, which is (2S)-(4E)-N-methyl-5-[3-(5-isopropoxypyridin)yl]-4-penten-2-amine, or pharmaceutically acceptable salts thereof, for the manufacturing of a medicament for use simultaneously, sequentially or separately, for the treatment of psychiatric disorders; particularly, cognitive impairment disorders in psychotic disorders.

A further embodiment of the invention relates to an agent comprising the combination comprising (a) a first therapeutic agent, which is an antipsychotic agent and (b) a second therapeutic agent, which is (2S)-(4E)-N-methyl-5-[3-(5-isopropoxypyridin)yl]-4-penten-2-amine, or pharmaceutically acceptable salts thereof, for use simultaneously, sequentially or separately, for treatment of psychiatric disorders; particularly, cognitive impairment disorders in psychotic disorders.

One embodiment relates to the methods as described above wherein the first therapeutic agent (a) comprises the group of compounds selected from chlorpromazine, haloperidol, flupenthixol, and perphenazine. Examples of atypical antipsychotics include but are not limited to clozapine, risperidone, olanzapine, quetiapine, aripiprazole, ziprasidone, amisulpride, sulpride, zotepine, sertindole, paliperidone, bifeprunox, and asenapine, metabolites or prodrugs and pharmaceutically acceptable salts, solvates or solvated salts thereof.

A further embodiment relates to the methods as described above wherein the antipsychotic agent (a) is an atypical antipsychotic agent.

Yet a further embodiment relates to the methods described above wherein the antipsychotic agent (a) is quetiapine, or pharmaceutically acceptable salts, solvates or solvated salts thereof.

One embodiment relates to a method for treating cognitive impairment and/or psychotic disorder in a subject in need thereof comprising administering simultaneously, sequentially or separately, to said subject a therapeutically effective amount of (a) a first therapeutic agent, which is quetiapine or pharmaceutically acceptable salts thereof and (b) a second therapeutic agent, which is (2S)-(4E)-N-methyl-5-[3-(5-isopropoxypyridin)yl]-4-penten-2-amine, or pharmaceutically acceptable salts thereof.

The invention further relates to methods for the treatment of Neurodegenerative Disorder(s) including, but are not limited to, Alzheimer's Disease (AD), Dementia, Cognitive Deficit in Schizophrenia (CDS), Mild Cognitive Impairment (MCI), Age-Associated Memory Impairment (AAMI), Age-Related Cognitive Decline (ARCD), Cognitive Impairment No Dementia (CIND), Multiple Sclerosis (MS), Parkinson's Disease (PD), postencephalitic parkinsonism, parkinsonism-dementia of Gaum, head trauma, Huntington's Disease, Amyotrophic Lateral Sclerosis (ALS), motor neuron diseases (MND), Multiple System Atrophy (MSA), Corticobasal Degeneration, Progressive Supranuclear Paresis, Guillain-Barré Syndrome (GBS), and Chronic Inflammatory Demyelinating Polyneuropathy (CIDP). Dementia includes, but is not limited to, Down syndrome, vascular dementia, dementia with Lewy bodies, HIV dementia, Frontotemporal dementia Parkinson's Type (FTDP), Pick's Disease, Niemann-Pick's Disease, traumatic brain injury (TBI), dementia pugilistica, Creutzfeld-Jacob Disease, and prion diseases.

The invention further relates to methods for the treatment of Neuroinflammatory Disorder(s) including, but not limited to, Multiple Sclerosis (MS), Parkinson's disease, Multiple System Atrophy (MSA), Corticobasal Degeneration, Progressive Supranuclear Paresis, Guillain-Barré Syndrome (GBS), chronic inflammatory demyelinating polyneuropathy (CIDP). Multiple sclerosis (MS) includes Relapse Remitting Multiple Sclerosis (RRMS), Secondary Progressive Multiple Sclerosis (SPMS), and Primary Progressive Multiple Sclerosis (PPMS).

The invention further relates to methods for the treatment of cognitive disorders, including, but not limited to:

a) Dementia, including, but not limited to, Alzheimer's Disease (AD), Down syndrome, vascular dementia, Parkinson's Disease (PD), postencephelatic parkinsonism, dementia with Lewy bodies, HIV dementia, Huntington's Disease, amyotrophic lateral sclerosis (ALS), motor neuron diseases (MND), Frontotemporal dementia Parkinson's Type (FTDP), progressive supranuclear palsy (PSP), Pick's Disease, Niemann-Pick's Disease, corticobasal degeneration, traumatic brain injury (TBI), dementia pugilistica, Creutzfeld-Jacob Disease, and prion diseases;

b) Cognitive Deficit in Schizophrenia (CDS);

c) Mild Cognitive Impairment (MCI);

d) Age-Associated Memory Impairment (AAMI);

e) Age-Related Cognitive Decline (ARCD);

f) Cognitive Impairment No Dementia (CIND); and

g) Addictions such as nicotine addiction.

The invention further relates to methods for the treatment of CNS disorder including, but not limited to, Attention-Deficit and Disruptive Behavior Disorder(s) including, but not limited to, attention deficit disorder (ADD), attention deficit hyperactivity disorder (ADHD), and affective disorders.

One embodiment of the invention relates to a method for treating CNS disorders wherein the cognitive disorder is dementia, dementia in Alzheimer's Disease, Cognitive Deficit in Schizophrenia (CDS), Mild Cognitive Impairment (MCI), Age-Associated Memory Impairment (AAMI), Age-Related Cognitive Decline (ARCD) or Cognitive Impairment No Dementia (CIND).

Another embodiment of the invention relates to a method for treating CNS disorders, wherein the disease is Cognitive Deficit in Schizophrenia.

The above conditions and disorders are defined for example in the American Psychiatric Association Diagnostic and Statistical Manual of Mental Disorders, Fourth Edition, Text Revision, Washington, D.C., American Psychiatric Association, 2000. This Manual may also be referred to for greater detail on the symptoms and diagnostic features associated with substance use, abuse and dependence.

The term “agent” means the compounds comprised in the combination of the present invention, i.e. an antipsychotic or an α4β2-neuronal nicotinic receptor agonist.

As used herein, an “agonist” is a substance that stimulates its binding partner, typically a receptor. Stimulation is defined in the context of the particular assay, or may be apparent in the literature from a discussion herein that makes a comparison to a factor or substance that is accepted as an “agonist” or an “antagonist” of the particular binding partner under substantially similar circumstances as appreciated by those of skill in the art. Stimulation may be defined with respect to an increase in a particular effect or function that is induced by interaction of the agonist or partial agonist with a binding partner and can include allosteric effects.

As used herein, a “partial agonist” is a substance that provides a level of stimulation to its binding partner that is intermediate between that of a full or complete antagonist and an agonist defined by any accepted standard for agonist activity. It will be recognized that stimulation, and hence, inhibition is defined intrinsically for any substance or category of substances to be defined as agonists, antagonists, or partial agonists.

“Cognitive impairment” includes, but is not limited to, an acquired deficit in one or more of memory function, problem solving, orientation and abstraction. “Cognitive function testing” may fall into the categories of attention related tasks such as simple reaction time, choice reaction time, and digit vigilance; categories of working memory such as numeric working memory and spatial working memory; categories of secondary episodic recognition memory testing, such as word recognition, picture recognition, immediate word recall, and delayed word recall; as well as other tasks such as visual tracking. Other examples of standard tests for measuring cognitive impairment may include, but are not limited to, the Mini Mental State Examination, the Global Deterioration Scale and Geriatric Depression Scale, the Randt Memory Test, and the Alzheimer's Disease Assessment Scale.

“Dementia” refers to global deterioration of intellectual functioning in clear consciousness, and is characterized by one or more symptoms of disorientation, impaired memory, impaired judgment, and/or impaired intellect. The symptoms of “dementia” are generally worse than, and can encompass, the symptoms of “cognitive impairment.”

“Cognitive impairments caused by traumatic brain injury” refers to cognitive impairments, as defined herein, that are associated with or caused by traumatic brain injuries, and other traumas to the head, such as, for example, traumas caused by accidents and/or sports injuries, including dementia pugilistica, which is severe brain damage caused by repeated blows to the head (e.g., from boxing). “Dementia pugilistica” is a chronic and progressive clinical syndrome characterized by neurological evidence of damage to pyramidal, extrapyramidal, and cerebellar systems with associated psychosis, dementia, personality change and impaired social functioning and/or prominent signs/symptoms of Parkinsonism (e.g., tremors, dysarthria, rigidity, bradykinesia, other extrapyramidal signs).

“Associated physical symptoms” may include inflammation-related aspects commonly associated with schizophrenia and may include conditions associated with elevated levels of inflammatory cytokines thought to modulate the symptomatology of schizophrenia, over-activation of the immune system by increases in the level of circulating monocytes or increased levels of Interleukin-6 (IL-6), necrotizing colitis, inflammation, edema and hemorrhage.

The phrase “therapeutically-effective amount” as used herein refers to a sufficient amount of the compound to treat CNS disorders or conditions at a reasonable risk/benefit ratio applicable to any medical treatment.

The term “treating” as used herein, refers to reversing, alleviating, inhibiting the progress of, or preventing the disorder or condition to which such term applies, or one or more symptoms of such disorder or condition. The term “treatment”, as used herein, refers to the act of “treating” as defined herein.

The term “disorder”, unless stated otherwise, has the same meaning as the terms “condition” and “disease” and are used interchangeably throughout the description and claims.

“Synergy” means an improved effect of the two agents in the combination, which is greater than the effect of the two agents together.

Dosage

The effective dose of an antipsychotic and an α4β2-neuronal nicotinic receptor agonist in the combinations according to the present invention may vary, depending upon factors such as the condition of the patient, the severity of the symptoms of the disorder as well as the potency of the selected specific compound, the mode of administration, the age and weight of the patient, and the like. Determining a dose is within the skill of the ordinary artisan.

Desirably, when quetiapine is selected as the antipsychotic agent, the daily dose of the combination contains from about 1 mg to about 1200 mg. Preferably, each dose of the first component contains about 25 mg to about 1000 mg of the quetiapine, and even more preferably, each dose contains from about 150 mg to about 800 mg or 300 mg to about 800 mg or 400 mg to about 800 mg of quetiapine. In another embodiment the first component contains about 150-300 or 300-600 mg of the quetiapine. Pediatric dosages may be less such as for example in the range of about 0.5 mg to about 40 mg daily. These dosages may be administered in one, two or more oral doses, for example: quetiapine: from about 1.0 to about 40 mg/kg given once daily or in divided doses.

One embodiment relates to the methods as described above wherein the quetiapine or pharmaceutically acceptable salt thereof is administered at a dosage of between about 5 mg to about 800 mg daily.

Another embodiment relates to the methods as described above wherein the quetiapine or pharmaceutically acceptable salt thereof is administered at a dosage of between about 10 mg to about 600 mg daily.

A further embodiment relates to the methods as described above wherein the quetiapine or pharmaceutically acceptable salt thereof is administered at a dosage of between about 25 mg to about 300 mg daily.

One embodiment relates to the methods as described above wherein the quetiapine or pharmaceutically acceptable salt thereof is administered at a dosage of between about 400 mg to about 800 mg daily.

Another embodiment of the invention relates to the methods as described above wherein the quetiapine or quetiapine salt is administered at a dosage of between about 150 mg to about 300 mg daily.

Typically, the effective dose of neuronal nicotinic receptor agonists generally requires administering the compound in an amount of less than 5 mg/kg of patient weight. Often, the neuronal nicotinic receptor agonists are administered in an amount from less than about 1 mg/kg patent weight to less than about 100 μg/kg of patient weight, and occasionally between about 10 μg/kg to less than 100 μg/kg of patient weight. The foregoing effective doses typically represent that amount administered as a single dose, or as one or more doses administered over a 24 hours period.

For human patients, the effective dose of the neuronal nicotinic receptor agonists generally requires administering the neuronal nicotinic receptor agonist in an amount of at least about 1, often at least about 10, and frequently at least about 25 mg/24 hours/patient. For human patients, the effective dose of the neuronal nicotinic receptor agonists requires administering the neuronal nicotinic receptor agonist which generally does not exceed about 500, often does not exceed about 400, and frequently does not exceed about 300 mg/24 hours/patient. The amount of neuronal nicotinic receptor agonists may be between 0.25 and 5 mg/24 hours/patient. In addition, administration of the effective dose is such that the concentration of the neuronal nicotinic receptor agonist within the plasma of the patient normally does not exceed 500 ng/mL, and frequently does not exceed 100 ng/mL.

One embodiment relates to a method for treating cognitive impairment and/or psychotic disorder, a method for improving the antipsychotic effect of quetiapine in reducing amphetamine-induced locomotor activity or a method of reducing sedative side effects of quetiapine whereby the dose of (2S)-(4E)-N-methyl-5-[3-(5-isopropoxypyridin)yl]-4-penten-2-amine is between 0.001 and 0.5 mg/kg.

One embodiment relates to a method for treating cognitive impairment and/or psychotic disorder, a method for improving the antipsychotic effect of quetiapine in reducing amphetamine-induced locomotor activity or a method of reducing sedative side effects of quetiapine whereby the dose of (2S)-(4E)-N-methyl-5-[3-(5-isopropoxypyridin)yl]-4-penten-2-amine is between 0.01 and 0.3 mg/kg.

A further embodiment relates to a method of inhibiting acoustic startle or a method of enhancing quetiapine-mediated reduction of phencyclidine-induced disruption of prepulse inhibition, whereby the dose of quetiapine is between 1 and 25 mg/kg.

Yet another embodiment relates to a method of inhibiting acoustic startle or a method of enhancing quetiapine-mediated reduction of phencyclidine-induced disruption of prepulse inhibition, whereby the dose of quetiapine is between 5 and 20 mg/kg.

U.S. provisional application Ser. No. 60/844,759 filed Sep. 15, 2006 is incorporated herein by reference in its entirety.

In order that the invention disclosed herein may be more efficiently understood, examples are provided below. It should be understood that these examples are for illustrative purposes only and are not to be construed as limiting the invention in any manner.

### EXAMPLES

### Example 1

### Prophetic

A pharmaceutical composition is prepared by combining quetiapine with a neuronal nicotinic receptor agonist in a pharmaceutically acceptable carrier. The composition contains respective amounts of quetiapine and the neuronal nicotinic receptor agonist to deliver on a daily basis a therapeutically-effective amount of each ingredient. The composition is administered to a patient for the treatment of cognitive impairment in association with schizophrenia on a daily, twice daily, three times daily, or four times daily basis.

### Example 2

### Prophetic

A pharmaceutical composition is prepared by combining quetiapine with (2S)-(4E)-N-methyl-5-[3-(5-isopropoxypyridin)yl]-4-penten-2-amine in a pharmaceutically acceptable carrier. The composition contains respective amounts of quetiapine and (2S)-(4E)-N-methyl-5-[3-(5-isopropoxypyridin)yl]-4-penten-2-amine to deliver on a daily basis a therapeutically-effective amount of each ingredient. The composition is administered to a patient for the treatment of cognitive impairment in association with schizophrenia on a daily, twice daily, three times daily, or four times daily basis.

### Example 3

### Biological Tests for Assessing Effects of Combinations of a Neuronal Nicotinic Receptor Agonist And an Antipsychotic Agent

The following examples from preclinical models are included for illustrative purposes should not be interpreted as restricting or limiting the invention in any way. A person with ordinary skill in the art will recognize related patient classes and other antipsychotic agents that could be substituted in the preclinical models provided.

Interaction design: Interactive effects of drug combinations are tested by co-administering varying doses of the neuronal nicotinic receptor agonist with the atypical antipsychotic drug and then conducting various biological tests (e.g. antipsychotic tests) to determine the existence of interactions (positive or negative). The dose range used for combination studies includes doses of the neuronal nicotinic receptor agonist known to be active in cognitive-enhancement tests (when tested alone) as well as doses below the threshold for cognitive activity (sub-threshold doses). Similarly, for the antipsychotic drug, the dose range includes doses in which the antipsychotic alone is active as well as sub-threshold doses.

I. Attenuation of Quetiapine-Induced Sedation Using Spontaneous Locomotor Activity (sLMA)

Quetiapine is known to have sedative properties in humans. Quetiapine can induce sedation in rats and is manifested in various behaviors including reductions in spontaneous locomotor activity (sLMA). A test compound that increases sLMA in quetiapine treated rats may reduce quetiapine-induced sedation in human subjects.

Procedures

The general procedure was to assess the effect of a particular drug on sLMA in rats by administering the drug after baseline assessment and then waiting an appropriate amount of time for the drug to achieve significant levels in plasma and brain. Previous studies with quetiapine showed that 15 minute pre-treatment at 40 mg/kg subcutaneous (s.c.) resulted in moderate, but significant, reductions in sLMA and this pre-treatment regimen was used to assess the ability of (2S)-(4E)-N-Methyl-5-[3-(5-isopropoxypyridin)yl]-4-penten-2-amine p-hydroxybenzoate (defined as Compound b hereafter) to attenuate quetiapine-induced sedation

Drug Preparation and Administration

Quetiapine was solubilized in saline plus some drops of 85% lactic acid and sonicated for around 20-30 minutes then adjusted with NaOH 1N for pH ˜4.8 to 5.0. The solution was stirred during the experiment to ensure uniform composition prior to administration. Quetiapine was administered by s.c. injection 15 minutes before testing. The dose volume was 4 ml/kg.

Compound b was dissolved in H₂O and administered by oral gavage (p.o.) 30 minutes before testing with a dose volume of 1 ml/kg.

The effect of Compound b alone on sLMA was tested by administering doses of 0.006, 0.019, 0.06 or 0.19 mg/kg by oral gavage (p.o.) 30 minutes before testing.

For combination studies of quetiapine and Compound b, immediately after baseline measurement, rats were removed from chambers and administered either: 1) vehicle 30 minutes before testing and quetiapine 15 minutes before testing, or 2) Compound b 30 minutes before testing and vehicle 15 minutes before testing, or 3) Compound b and quetiapine at 30 minutes and 15 minutes respectively before testing, or 4) vehicle and vehicle at 30 minutes and 15 minutes respectively before testing. During the periods between drug administration and testing, rats were returned to the home cages.

Apparatus

Exploratory activity was monitored and quantified using open field activity systems (hardware and software) purchased from Med Associates.

Subjects

Subjects were male, Long Evans rats weighing 300-350 g and fasted overnight. Rats were acclimated in the laboratory at least 1 hour prior to testing. Rats were weighed and placed into activity chambers for 5 minutes to measure baseline activity.

Statistical Analysis

Statistical analysis was made of total distance traveled over the 5 minutes test period for a spontaneous locomotor activity using one-way analysis ANOVA and Turkey's multiple-range analysis. All values are expressed as mean and SD.

Results

1. Effect of Compound b at 0.006, 0.019, 0.06 and 0.19 mg/kg, p.o. on sLMA

Compound b had no significant effect on sLMA at any dose tested. All rats demonstrated a normal locomotor activity over 5 minute baseline period (FIG. 1).

2. Effect of Compound b on Quetiapine-Induced Sedation

For the combination study of the effects of Compound b on quetiapine-induced sedation study, Compound b was administered at 0.19 mg/kg p.o. and quetiapine was administered at 40 mg/kg s.c. Compared to administration of vehicle and vehicle rats administered vehicle and quetiapine 40 mg/kg showed a significantly reduction in distance traveled and thus evidence of quetiapine-induced sedation. When administered before quetiapine, 0.19 mg/kg of Compound b did not result in a significant reduction in sLMA compared to vehicle-vehicle controls supporting the hypothesis that Compound b reduced quetiapine-induced sedation in this model. Furthermore, rats administered Compound b and then vehicle showed no significant suppression of sLMA confirming results from experiment 1 that the compound has no significant suppressant or stimulant properties.

II. Enhancement of Quetiapine Blockade of Amphetamine-Induced Increases in Locomotor Activity (LMA)

Blockade of amphetamine-induced locomotor activity model: Amphetamine can induce psychotic reactions in humans, typically after relatively high doses or chronic use.

Amphetamine can worsen the symptoms of schizophrenia. Antipsychotic drugs are well known to block many of the effects of amphetamine in experimental animals and humans. In the D-amphetamine-induced LMA test, established antipsychotic drugs have been extensively shown to block D-amphetamine-induced increases in LMA (as manifested in various behaviors including locomotor activity). A test compound that enhanced the effect of a known antipsychotic drug in this model but which had no blocking of amphetamine effect when administered alone would support the hypothesis that the test compound may enhance antipsychotic effects of the known antipsychotic drug in human patients.

Procedures

The general procedure for the amphetamine model is to allow each subject to habituate to an activity chamber for 60 minutes, inject them with a dose of D-amphetamine that induces a high level of locomotor exploratory activity and then measure the locomotor activity for 60 minutes. Antipsychotic drugs and other test compounds are administered at appropriate pre-treatment times before the D-amphetamine (typically during the habituation period).

Drug Preparation and Administration

Quetiapine was solubilized in saline plus some drops of 85% lactic acid and sonicated for around 20-30 minutes then adjusted with NaOH 1N for pH 4.8 to 5.0. The solution was stirred during the experiment to ensure uniform composition prior to administration. Quetiapine was administered by subcutaneous injection 15 minutes before administration of D-amphetamine. The dose volume was 4 ml/kg. For dose-response analysis, quetiapine was administered at 10, 20, 40 or 60 mg/kg s.c. For combination studies, quetiapine was administered at 10 mg/kg s.c.

Compound b was dissolved in H₂O and administered by oral gavage (p.o.) 30 minutes before D-amphetamine with a dose volume of 1 ml/kg.

D-amphetamine was dissolved in phosphate buffered saline (PBS) and administed at 1 mg/kg by subcutaneous injection in a volume 1 ml/kg immediately before the start of the test session.

Subjects

Subjects were male Long Evans rats weighing 300-350 g and fasted overnight. Rats were acclimated in the laboratory at least 1 hour prior to testing.

Apparatus

Exploratory activity was monitored and quantified using open field activity systems (hardware and software) purchased from Med Associates.

Statistical Analysis

Statistical analysis was made of total distance traveled after D-amphetamine administration using ANOVA and Tukey's post hoc analysis where appropriate. All values are expressed as mean and SD.

Results

1. Dose Response of Quetiapine for Reduction of D-Amphetamine-Induced Locomotor Activity

Amphetamine significantly increased locomotor activity in habituated rats, p<0.001.

Quetiapine significantly attenuated amphetamine-induced locomotor activity at doses of 20, 40 and 60 mg/kg but not 10 mg/kg (FIG. 3). The threshold for significantly reducing D-amphetamine-induced locomotor activity was 20 mg/kg.

2. Effect of the Combination of Compound B at 0.019, 0.06 and 0.19 mg/kg (p.o.) and Quetiapine at 10 mg/kg (s.c.) on D-Amphetamine-Induced the Locomotor Activity.

Amphetamine significantly increased locomotor activity in habituated rats, p<0.001. Administration of quetiapine at 10 mg/kg sc did not significantly attenuate amphetamine-induced the locomotor activity. Administration of Compound b at 0.19 mg/kg also did not significantly reduce D-amphetamine-induced locomotor activity. Administration of a combination of quetiapine 10 mg/kg and Compound b at 0.019 or 0.06 mg/kg p.o. did not significantly reduce D-amphetamine-induced locomotor activity. However, administration of Compound b at 0.19 mg/kg p.o. with quetiapine at 10 mg/kg s.c. did significantly reduce D-amphetamine-induced locomotor activity compared to the D-amphetamine alone group. These results support the hypothesis that Compound b can enhance the antipsychotic effects of quetiapine.

III. Blockade of Haloperidol-Induced Catalepsy

Haloperidol-induced catalepsy model of EPS: Haloperidol is a typical or first-generation antipsychotic drug that while effective is prone to produce extrapyramidal symptoms (EPS) in humans. Other antipsychotic drugs (e.g. chlorpromazine, risperidone) can also cause EPS in patients and this side effect can limit the use of these drugs. In rodents, drug-induced catalepsy (a trance-like state of waxy immobility) can be used to assess the risk of EPS for antipsychotic drugs. Haloperidol reliably induces catalepsy in mice and rats. Test compounds that reduce catalepsy in haloperidol-treated rats may reduce antipsychotic drug-induced EPS.

Procedure

Haloperidol-induced catalepsy in mice model: CF-1 male mice (Charles River, 20-30 gm, 10 mice per dose) were dosed orally with compound (b) at 0.06, 0.19, 0.6 and 1.9 mg/kg or vehicle (distilled water). Thirty minutes later, all mice were dosed with 2 mg/kg of haloperidol s.c. At 60 minutes and 4 hours after haloperidol dosing, the experimenter gently placed both forepaws of each animal on a metal bar (4 mm in diameter) that was fixed horizontally 5 cm above the test floor. The length of time (in seconds) during which each mouse maintained the initial forepaw bar position was recorded (cataleptic pose). Maximum cut-off time was 60 seconds. The same experimental design was performed on two consecutive days, and results were combined from both experiments. Therefore, the final results are from groups of 20 mice.

Results

Haloperidol at 2 mg/kg reliably produces catalepsy of up to 60 seconds in 60-90% of mice. In contrast, pre-treatment with Compound b reduced this haloperidol induced phenotype (FIG. 5). At the 60 minute time point, Compound b reduced haloperidol induced catalepsy at all 4 concentrations tested (P<0.05, One-way ANOVA followed by Dunnett's Multiple Comparision Test). At the 4 hour time point, only the 1.9 mg/kg dose of compound (b) caused a significant reduction of haloperidol induced catalepsy.

### CONCLUSION

Compound b has the capacity to attenuate haloperidol induced catalepsy in CF-1 mice. The results for Compound b are surprising and unexpected for several reasons. First, Compound b did not have stimulant properties as demonstrated in the sLMA studies (FIG. 1) and therefore simple stimulant effects do not explain the ability of Compound b to reduce haloperidol-induced catalepsy. Secondly, there are prior studies demonstrating that the non-selective nicotinic agonist nicotine can worsen catalepsy in rodent (for example Sanberg P., et al., Pharm. Biochem. Behav., vol 46, p 303-307, 1993). Another study showed that while nicotine and another nicotinic agonist RJR 2403 did not significantly worsen haloperidol-induced catalepsy, they did not reduce it either (Levin E. and Lippiello P., Drug Devel. Res., 47:90-96, 1999). Therefore, it is surprising and unexpected that the selective α4β2-neuronal nicotinic receptor agonists Compound b should reduce haloperidol-induced catalepsy.

Various modifications of the invention, in addition to those described herein, will be apparent to those skilled in the art from the foregoing description. Such modifications are also intended to fall within the scope of the appended claims. Each reference (including, but not limited to, journal articles, U.S. and non-U.S. patents, patent application publications, international patent application publications, and the like) cited in the present application is incorporated herein by reference in its entirety.

What is claimed is:

1. A combination comprising:
an antipsychotic agent, or a pharmaceutically acceptable salt thereof, and
(2S)-(4E)-N-methyl-5-[3-(5-isopropoxypyridin)yl]-4-penten-2-amine, or a pharmaceutically acceptable salt thereof.

2. The combination according to claim 1 wherein the antipsychotic agent is quetiapine or a pharmaceutically acceptable salt thereof.

3. A pharmaceutical composition comprising a combination comprising:
an antipsychotic agent, or a pharmaceutically acceptable salt thereof; and
(2S)-(4E)-N-methyl-5-[3-(5-isopropoxypyridin)yl]-4-penten-2-amine, or a pharmaceutically acceptable salt thereof;
together with a pharmaceutically acceptable vehicle, carrier, or diluent.

4. The pharmaceutical composition according to claim 3 wherein the antipsychotic agent is quetiapine or a pharmaceutically acceptable salt thereof.

5. A combination comprising:
an antipsychotic agent, or a pharmaceutically acceptable salt thereof; and
an α4β2-selective neuronal nicotinic receptor agonist, or a pharmaceutically acceptable salt thereof.

6. A pharmaceutical composition comprising a combination comprising:
antipsychotic agent, or a pharmaceutically acceptable salt thereof; and
an α4β2-selective neuronal nicotinic receptor agonist, or a pharmaceutically acceptable salt thereof;
together with a pharmaceutically acceptable vehicle, carrier, or diluent.

7. A kit comprising:
a dosage unit of an antipsychotic agent, or a pharmaceutically acceptable salt thereof; and
a dosage unit of (2S)-(4E)-N-methyl-5-[3-(5-isopropoxypyridin)yl]-4-penten-2-amine, or a pharmaceutically acceptable salt thereof;
optionally with instructions for use.

8. The kit according to claim 7 wherein the antipsychotic agent is quetiapine or a pharmaceutically acceptable salt thereof.

9. A method for treating cognitive impairment and/or psychotic disorder in a subject in need thereof comprising administering simultaneously, sequentially or separately, to said subject a therapeutically effective amount of an antipsychotic agent, or a pharmaceutically acceptable salt thereof, and (2S)-(4E)-N-methyl-5-[3-(5-isopropoxypyridin)yl]-4-penten-2-amine, or a pharmaceutically acceptable salt thereof.

10. The method according to claim 9 whereby the psychotic disorder is selected from schizophrenia, schizophreniform disorder, schizoaffective disorder, delusional disorder, brief psychotic disorder, treatment-resistant shared psychotic disorder, and psychotic disorder due to a medical condition.

11. The method according to claim 9 wherein the antipsychotic agent and (2S)-(4E)-N-methyl-5-[3-(5-isopropoxypyridin)yl]-4-penten-2-amine, or pharmaceutically acceptable salts thereof, are administered simultaneously, sequentially or separately, to the subject in a pharmaceutical composition additionally comprising a pharmaceutically acceptable vehicle, carrier, or diluent.

12. The method according to claim 10 wherein the antipsychotic agent and (2S)-(4E)-N-methyl-5-[3-(5-isopropoxypyridin)yl]-4-penten-2-amine, or pharmaceutically acceptable salts thereof, are administered simultaneously, sequentially or separately, to the subject in a pharmaceutical composition additionally comprising a pharmaceutically acceptable vehicle, carrier, or diluent.

13. The method according to claim 9 wherein the antipsychotic agent is quetiapine, or a pharmaceutically acceptable salt thereof.

14. The method according to claim 9 wherein the dose of quetiapine is between 5 and 50 mg/kg.

15. The method according to claim 9 wherein the dose of quetiapine is between 10 and 40 mg/kg.

16. The method according to claim 9 wherein the dose of (2S)-(4E)-N-methyl-5-[3-(5-isopropoxypyridin)yl]-4-penten-2-amine is between 0.001 and 0.5 mg/kg.

17. The method according to claim 9 wherein the dose of (2S)-(4E)-N-methyl-5-[3-(5-isopropoxypyridin)yl]-4-penten-2-amine is between 0.01 and 0.3 mg/kg.

18. A method for improving the antipsychotic effect of quetiapine in a subject in need thereof comprising administering simultaneously, sequentially or separately, to said subject an antipsychotic agent, or a pharmaceutically acceptable salt thereof, and (2S)-(4E)-N-methyl-5-[3-(5-isopropoxypyridin)yl]-4-penten-2-amine, or a pharmaceutically acceptable salt thereof.

19. The method according to claim 18 wherein the antipsychotic agent is quetiapine, or a pharmaceutically acceptable salt thereof.

20. The method according to claim 18 wherein the dose of quetiapine is between 5 and 50 mg/kg.

21. The method according to claim 18 wherein the dose of quetiapine is between 10 and 40 mg/kg.

22. The method according to claim 18 wherein the dose of (2S)-(4E)-N-methyl-5-[3-(5-isopropoxypyridin)yl]-4-penten-2-amine is between 0.001 and 0.5 mg/kg.

23. The method according to claim 18 wherein the dose of (2S)-(4E)-N-methyl-5-[3-(5-isopropoxypyridin)yl]-4-penten-2-amine is between 0.01 and 0.3 mg/kg.

24. A method of increasing dopamine release in prefrontal cortex in a subject in need thereof comprising administering simultaneously, sequentially or separately, to said subject an antipsychotic agent, or a pharmaceutically acceptable salt thereof, and (2S)-(4E)-N-methyl-5-[3-(5-isopropoxypyridin)yl]-4-penten-2-amine, or a pharmaceutically acceptable salt thereof.

25. The method according to claim 24 wherein the antipsychotic agent is quetiapine, or a pharmaceutically acceptable salt thereof.

26. The method according to claim 24 wherein the dose of quetiapine is between 5 and 50 mg/kg.

27. The method according to claim 24 wherein the dose of quetiapine is between 10 and 40 mg/kg.

28. The method according to claim 24 wherein the dose of (2S)-(4E)-N-methyl-5-[3-(5-isopropoxypyridin)yl]-4-penten-2-amine is between 0.001 and 0.5 mg/kg.

29. The method according to claim 24 wherein the dose of (2S)-(4E)-N-methyl-5-[3-(5-isopropoxypyridin)yl]-4-penten-2-amine is between 0.01 and 0.3 mg/kg.

30. A method of reducing a sedative side effect of quetiapine in a subject in need thereof comprising administering simultaneously, sequentially or separately, to said subject a therapeutically effective amount of an antipsychotic agent, or a pharmaceutically acceptable salt thereof, and (2S)-(4E)-N-methyl-5-[3-(5-isopropoxypyridin)yl]-4-penten-2-amine, or a pharmaceutically acceptable salt thereof.

31. The method according to claim 30 wherein the antipsychotic agent is quetiapine, or a pharmaceutically acceptable salt thereof.

32. The method according to claim 30 wherein the dose of quetiapine is between 5 and 50 mg/kg.

33. The method according to claim 30 wherein the dose of quetiapine is between 10 and 40 mg/kg.

34. The method according to claim 30 wherein the dose of (2S)-(4E)-N-methyl-5-[3-(5-isopropoxypyridin)yl]-4-penten-2-amine is between 0.001 and 0.5 mg/kg.

35. The method according to claim 30 wherein the dose of (2S)-(4E)-N-methyl-5-[3-(5-isopropoxypyridin)yl]-4-penten-2-amine is between 0.01 and 0.3 mg/kg.

36. A method of inhibiting acoustic startle in a subject in need thereof comprising administering simultaneously, sequentially or separately, to said subject an antipsychotic agent, or a pharmaceutically acceptable salt thereof, and (2S)-(4E)-N-methyl-5-[3-(5-isopropoxypyridin)yl]-4-penten-2-amine, or a pharmaceutically acceptable salt thereof.

37. The method according to claim 36 wherein the antipsychotic agent is quetiapine or a pharmaceutically acceptable salt thereof.

38. The method according to claim 36 wherein the dose of quetiapine is between 1 and 25 mg/kg.

39. The method according to claim 36 wherein the dose of quetiapine is between 5 and 20 mg/kg.

40. The method according to claim 36 wherein the dose of (2S)-(4E)-N-methyl-5-[3-(5-isopropoxypyridin)yl]-4-penten-2-amine is between 0.001 and 0.05 mg/kg.

41. The method according to claim 36 wherein the dose of (2S)-(4E)-N-methyl-5-[3-(5-isopropoxypyridin)yl]-4-penten-2-amine is between 0.01 and 0.03 mg/kg.

42. A method of enhancing quetiapine-mediated reduction of phencyclidine-induced disruption of prepulse inhibition in a subject in need thereof comprising administering simultaneously, sequentially or separately, to said subject an antipsychotic agent, or a pharmaceutically acceptable salt thereof, and (2S)-(4E)-N-methyl-5-[3-(5-isopropoxypyridin)yl]-4-penten-2-amine, or a pharmaceutically acceptable salt thereof.

43. The method according to claim 42 wherein the antipsychotic agent is quetiapine or a pharmaceutically acceptable salt thereof.

44. The method according to claim 42 wherein the dose of quetiapine is between 1 and 25 mg/kg.

45. The method according to claim 42 wherein the dose of quetiapine is between 5 and 20 mg/kg.

46. The method according to claim 42 wherein the dose of (2S)-(4E)-N-methyl-5-[3-(5-isopropoxypyridin)yl]-4-penten-2-amine is between 0.001 and 0.05 mg/kg.

47. The method according to claim 42 wherein the dose of (2S)-(4E)-N-methyl-5-[3-(5-isopropoxypyridin)yl]-4-penten-2-amine is between 0.01 and 0.03 mg/kg.

48. A method of reducing haloperidol-induced catalepsy in a subject in need thereof comprising administering simultaneously, sequentially or separately, to said subject haloperidol, or a pharmaceutically acceptable salt thereof, and (2S)-(4E)-N-methyl-5-[3-(5-isopropoxypyridin)yl]-4-penten-2-amine, or a pharmaceutically acceptable salt thereof.

49. A method of treating cognitive impairment and psychotic disorder in a subject in need thereof comprising administering simultaneously, sequentially or separately, to said subject an amount an antipsychotic agent, or a pharmaceutically acceptable salt thereof, and an amount of an α4β2 selective neuronal nicotinic receptor agonist, or a pharmaceutically acceptable salt thereof.
